(19) United States
(12) Patent Application Publication
(10) Pub. No.: US 2011/0306139 A1
(43) Pub. Date: Dec. 15, 2011

(54) Mutant Cells Suitable For Recombinant Polypeptide Production

(75) Inventors: Allan Kent Nielsen, Soeborg (DK); Michael Dolberg Rasmussen, Vallenbaek (DK); Niels Banke, Soeborg (DK)

(73) Assignee: Novozymes A/S, Bagsvaerd (DK)

(21) Appl. No.: 13/203,050

(22) PCT Filed: Feb. 25, 2010
(86) PCT No.: PCT/EP2010/052409

§ 371 Date: Aug. 31, 2011

(30) Foreign Application Priority Data
Feb. 27, 2009 (EP) 09153894.2

(51) Int. Cl. C12N 15/75; C12N 9/88; C12N 9/90; C12N 9/14; C12N 9/02; C12N 9/10; C12N 1/21; C12N 9/00

(52) U.S. Cl. 435/471; 435/252.31; 435/232; 435/183; 435/195; 435/189; 435/193; 435/233

(57) ABSTRACT

A mutated bacterial cell producing at least one heterologous polypeptide of interest, wherein said cell has a reduced expression-level of a polypeptide comprising an amino acid sequence at least 70% identical to the sequence shown in SEQ ID NO: 2, when compared with an otherwise isogenic but non-mutated cell; methods for producing said mutated cell; and methods for producing a polypeptide of interest using said mutated cell. SEQ ID NO: 2 represents a putative metalloprotease.

### SEQUENCE LISTING

The present invention comprises a sequence listing.

### FIELD OF THE INVENTION

The invention relates to a mutated bacterial cell producing at least one heterologous polypeptide of interest, wherein said cell has a reduced expression-level of a polypeptide comprising an amino acid sequence at least 70% identical to the sequence shown in SEQ ID NO: 2, when compared with an otherwise isogenic but non-mutated cell; methods for producing said mutated cell; and methods for producing a polypeptide of interest using said mutated cell.

### BACKGROUND OF THE INVENTION

The recombinant industrial manufacture of polypeptides, in particular, enzymes, is a very competitive area where the level of skill is extremely high. Industrial production of polypeptides in Bacillus is especially well-researched and, today, even incremental improvements of polypeptide yield or productivity are desirable in this field.

It has been known for years that the inactivation of extracellular and cell-wall associated proteases in Bacillus in many cases leads to improved product stability and therefore better yields (WO 1992/016642). As many as six proteases have inactivated in the Bacillus subtilis strain WB600 (Wu et al. 1991. Engineering a Bacillus subtilis expression-secretion system with a strain deficient in six extracellular proteases. J Bacteriol 173(16): 4952-4958).

The open reading frame (ORF) shown in SEQ ID NO:1 encodes a putative metalloprotease denoted BL00829, the amino acid sequence of which is shown in SEQ ID NO:2 (NCBI “Entrez Protein” or “Entrez Gene” accession number: YP_{—}080660), which was originally predicted from the genome sequencing of Bacillus licheniformis ATCC14580 by Rey et al in 2004: Complete genome sequence of the industrial bacterium Bacillus licheniformis and comparisons with closely related Bacillus species, Genome Biol. (10):R77. To our knowledge, no research on the predicted ORF or the putative encoded metalloprotease activity has been published since.

### SUMMARY OF THE INVENTION

Inactivation of the putative open reading frame shown in SEQ ID NO:1 in several Bacillus licheniformis enzyme production strains lead to significantly improved enzyme yields, as shown in the examples. The host strains had already had the two major extracellular proteases inactivated, the alkaline and neutral proteases (apr, npr), as well as the C-component (BLC). These inactivations eliminated by far the major part (>90%) of extracellular protease activity.

Inactivation of yet another protease would, at best, be expected to provide only a very minor improvement. In light of the above, it was highly surprising that the inactivation of the putative metalloprotease of the invention provided such a significantly improved product yield as seen in the examples.

Accordingly, in a first aspect the invention relates to mutated bacterial cell producing at least one heterologous polypeptide of interest, wherein said cell has a reduced expression-level of a polypeptide comprising an amino acid sequence at least 70% identical to the sequence shown in SEQ ID NO: 2, or preferably at least 75%, 80%, 85%, 90%, 92%, 94%, 96%, 98%, or 99% identical to SEQ ID NO: 2, when compared with an otherwise isogenic but non-mutated cell.

A second aspect of the invention relates to a method for constructing a mutated bacterial cell, said method comprising the steps of: a) mutating a bacterial cell; and b) selecting a mutated cell which has a reduced expression-level of a polypeptide comprising an amino acid sequence at least 70% identical to the sequence shown in SEQ ID NO: 2, or preferably at least 75%, 80%, 85%, 90%, 92%, 94%, 96%, 98%, or 99% identical to SEQ ID NO: 2, when compared with an otherwise isogenic but non-mutated cell.

A final aspect of the invention relates to a method for producing a heterologous polypeptide of interest, said method comprising the steps of: a) cultivating a mutated bacterial cell producing at least one heterologous polypeptide of interest, wherein said mutated cell has a reduced expression-level of a polypeptide comprising an amino acid sequence at least 70% identical to the sequence shown in SEQ ID NO: 2, or preferably at least 75%, 80%, 85%, 90%, 92%, 94%, 96%, 98%, or 99% identical to SEQ ID NO: 2, when compared with an otherwise isogenic but non-mutated cell, and b) isolating the polypeptide of interest.

### BRIEF DESCRIPTION OF DRAWINGS

FIG. 1 shows a schematic of plasmid pMDT081.

FIG. 2 shows a schematic of plasmid pAN829.

FIG. 3 shows a schematic of plasmid pMOL2598.

FIG. 4 shows a schematic of plasmid pMOL2606.

FIG. 5 shows a schematic of plasmid pAN369.

FIG. 6 shows a schematic of plasmid pAN405.

### DEFINITIONS

Isolated polypeptide: The term “isolated polypeptide” as used herein refers to a polypeptide that is isolated from a source. In a preferred aspect, the polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE.

Substantially pure polypeptide: The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides of the present invention are preferably in a substantially pure form, i.e., that the polypeptide preparation is essentially free of other polypeptide material with which it is natively or recombinantly associated. This can be accomplished, for example, by preparing the polypeptide by well-known recombinant methods or by classical purification methods.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Isolated polynucleotide: The term “isolated polynucleotide” as used herein refers to a polynucleotide that is isolated from a source. In a preferred aspect, the polynucleotide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by agarose electrophoresis.

Substantially pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form, i.e., that the polynucleotide preparation is essentially free of other polynucleotide material with which it is natively or recombinantly associated. The polynucleotides may be of genomic, cDNA, RNA, semisynthetic, synthetic origin, or any combinations thereof.

Coding sequence: When used herein the term “coding sequence” or “open reading frame” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant nucleotide sequence.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Control sequences: The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” denotes herein a configuration in which a control sequence is placed at an appropriate position relative to the coding sequence of the polynucleotide sequence such that the control sequence directs the expression of the coding sequence of a polypeptide.

Expression: The term “expression” includes any step involved in the production of the polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide of the present invention and is operably linked to additional nucleotides that provide for its expression.

Host cell: The term “host cell”, as used herein, includes any cell type that is susceptible to transformation, transfection, transduction, and the like with a nucleic acid construct or expression vector comprising a polynucleotide of the present invention. Modification: The term “modification” means herein any chemical modification of the polypeptide consisting of the mature polypeptide of SEQ ID NO: 2; or a homologous sequence thereof; as well as genetic manipulation of the DNA encoding such a polypeptide. The modification can be a substitution, a deletion and/or an insertion of one or more (several) amino acids as well as replacements of one or more (several) amino acid side chains.

Hybridization: For purposes of the present invention, hybridization indicates that the nucleotide sequence hybridizes to a labeled nucleic acid probe corresponding to the polypeptide coding sequence of SEQ ID NO: 1; the mature polypeptide coding sequence of SEQ ID NO: 1; its full-length complementary strand; or a subsequence thereof; under very low to very high stringency conditions. Molecules to which the nucleic acid probe hybridizes under these conditions can be detected using, for example, X-ray film. In a preferred aspect, the nucleic acid probe is the mature polypeptide coding sequence of SEQ ID NO: 1. In another preferred aspect, the nucleic acid probe is a polynucleotide sequence that encodes the polypeptide of SEQ ID NO: 2, or a subsequence thereof of at least 100 bp. In another preferred aspect, the nucleic acid probe is SEQ ID NO: 1.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at 45° C. (very low stringency), more preferably at 50° C. (low stringency), more preferably at 55° C. (medium stringency), more preferably at 60° C. (medium-high stringency), even more preferably at 65° C. (high stringency), and most preferably at 70° C. (very high stringency).

Mutation: Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127). Standard gene disruptions by frame-shift or partial or even full deletion may also be employed.

The present invention also relates to mutant polynucleotides comprising or consisting of at least one mutation in the mature polypeptide coding sequence of SEQ ID NO: 1, in which the mutant nucleotide sequence encodes the mature polypeptide of SEQ ID NO: 2.

The techniques used to isolate or clone a polynucleotide encoding a polypeptide are known in the art and include isolation from genomic DNA, preparation from cDNA, or a combination thereof. The cloning of the polynucleotides of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from a strain of Bacillus, or another or related organism and thus, for example, may be an allelic or species variant of the polypeptide encoding region of the nucleotide sequence.

The present invention also relates to methods of producing a mutant of a parent cell, which comprises disrupting or deleting a polynucleotide sequence, or a portion thereof, encoding a polypeptide of the present invention, which results in the mutant cell producing less of the polypeptide than the parent cell when cultivated under the same conditions.

The mutant cell may be constructed by reducing or eliminating expression of a nucleotide sequence encoding a polypeptide of the present invention using methods well known in the art, for example, insertions, disruptions, replacements, or deletions. In a preferred aspect, the nucleotide sequence is inactivated. The nucleotide sequence to be modified or inactivated may be, for example, the coding region or a part thereof essential for activity, or a regulatory element required for the expression of the coding region. An example of such a regulatory or control sequence may be a promoter sequence or a functional part thereof, i.e., a part that is sufficient for affecting expression of the nucleotide sequence. Other control sequences for possible modification include, but are not limited to, a leader, polyadenylation sequence, propeptide sequence, signal peptide sequence, transcription terminator, and transcriptional activator.

Modification or inactivation of the nucleotide sequence may be performed by subjecting the parent cell to mutagenesis and selecting for mutant cells in which expression of the nucleotide sequence has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of the gene.

Modification or inactivation of the nucleotide sequence may be accomplished by introduction, substitution, or removal of one or more (several) nucleotides in the gene or a regulatory element required for the transcription or translation thereof. For example, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the nucleotide sequence to be modified, it is preferred that the modification be performed in vitro as exemplified below.

An example of a convenient way to eliminate or reduce expression of a nucleotide sequence by a cell is based on techniques of gene replacement, gene deletion, or gene disruption. For example, in the gene disruption method, a nucleic acid sequence corresponding to the endogenous nucleotide sequence is mutagenized in vitro to produce a defective nucleic acid sequence that is then transformed into the parent cell to produce a defective gene. By homologous recombination, the defective nucleic acid sequence replaces the endogenous nucleotide sequence. It may be desirable that the defective nucleotide sequence also encodes a marker that may be used for selection of transformants in which the nucleotide sequence has been modified or destroyed. In a particularly preferred aspect, the nucleotide sequence is disrupted with a selectable marker such as those described herein.

Alternatively, modification or inactivation of the nucleotide sequence may be performed by established anti-sense or RNAi techniques using a sequence complementary to the nucleotide sequence. More specifically, expression of the nucleotide sequence by a cell may be reduced or eliminated by introducing a sequence complementary to the nucleotide sequence of the gene that may be transcribed in the cell and is capable of hybridizing to the mRNA produced in the cell. Under conditions allowing the complementary anti-sense nucleotide sequence to hybridize to the mRNA, the amount of protein translated is thus reduced or eliminated.

The present invention further relates to a mutant cell of a parent cell that comprises a disruption or deletion of a nucleotide sequence encoding the polypeptide or a control sequence thereof, which results in the mutant cell producing less of the polypeptide or no polypeptide compared to the parent cell.

The polypeptide-deficient mutant cells so created are particularly useful as host cells for the expression of native and/or heterologous polypeptides. Therefore, the present invention further relates to methods of producing a native or heterologous polypeptide comprising: (a) cultivating the mutant cell under conditions conducive for production of the polypeptide; and (b) recovering the polypeptide. The term “heterologous polypeptides” is defined herein as polypeptides that are not native to the host cell, a native protein in which modifications have been made to alter the native sequence, or a native protein whose expression is quantitatively altered as a result of a manipulation of the host cell by recombinant DNA techniques.

Expression Vectors The present invention also relates to recombinant expression vectors comprising a polynucleotide encoding the polypeptide of interest, a promoter, and transcriptional and translational stop signals. The various nucleic acids and control sequences described herein may be joined together to produce a recombinant expression vector that may include one or more (several) convenient restriction sites to allow for insertion or substitution of the nucleotide sequence encoding the polypeptide at such sites. Alternatively, a polynucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vectors of the present invention preferably contain one or more (several) selectable markers that permit easy selection of transformed, transfected, transduced, or the like cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers that confer antibiotic resistance such as ampicillin, kanamycin, chloramphenicol, or tetracycline resistance.

The vectors of the present invention preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity to the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” is defined herein as a nucleotide sequence that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

The procedures used to ligate the elements described above to construct the recombinant expression vectors of the present invention are well known to one skilled in the art (see, e.g., Sambrook et al., 1989, supra).

Host Cells: The present invention also relates to recombinant host cells, comprising an isolated polynucleotide of the present invention, which are advantageously used in the recombinant production of the polypeptides. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram positive bacterium or a Gram negative bacterium. Gram positive bacteria include, but not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but not limited to, E. coli, Pseudomonas, Salmonella, Campylobacter, Helicobacter, Flavobacterium, Fusobacterium, Ilyobacter, Neisseria, and Ureaplasma.

The bacterial host cell may be any Bacillus cell. Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

In a preferred aspect, the bacterial host cell is a Bacillus amyloliquefaciens, Bacillus lentus, Bacillus licheniformis, Bacillus stearothermophilus or Bacillus subtilis cell. In a more preferred aspect, the bacterial host cell is a Bacillus amyloliquefaciens cell. In another more preferred aspect, the bacterial host cell is a Bacillus clausii cell. In another more preferred aspect, the bacterial host cell is a Bacillus licheniformis cell. In another more preferred aspect, the bacterial host cell is a Bacillus subtilis cell. The introduction of DNA into a Bacillus cell may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). The introduction of DNA into an E coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57).

Methods of Production: The present invention also relates to methods of producing a recombinant polypeptide, comprising: (a) cultivating a mutated bacterial cell, which produces the polypeptide, under conditions conducive for production of the polypeptide; and (b) recovering the polypeptide. In a preferred aspect, the cell is of the genus Bacillus. In a more preferred aspect, the cell is Bacillus licheniformis.

In the production methods of the present invention, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted into the medium, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure polypeptides.

### DETAILED DESCRIPTION OF THE INVENTION

### Microorganisms

The microorganism (microbial strain or cell) according to the invention may be obtained from microorganisms of any genus, such as those bacterial sources listed below. In a preferred embodiment the cell of the first aspects of the invention is a prokaryotic cell, preferably a Gram-positive cell, more preferably a Bacillus cell, and most preferably a Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, or Bacillus thuringiensis cell.

### The Mutated Cell

In a preferred embodiment of the invention, the polypeptide comprising an amino acid sequence at least 70% identical to the sequence shown in SEQ ID NO: 2 or preferably at least 75%, 80%, 85%, 90%, 92%, 94%, 96%, 98%, or 99% identical to SEQ ID NO: 2, is a metallopeptidase.

In another preferred embodiment the mutated cell of the invention is mutated in a gene encoding the polypeptide comprising an amino acid sequence at least 70% identical to the sequence shown in SEQ ID NO: 2, preferably at least 75%, 80%, 85%, 90%, 92%, 94%, 96%, 98%, or 99% identical to SEQ ID NO: 2; preferably the mutated cell of the invention is mutated in a polynucleotide having a nucleotide sequence at least 70% identical to the sequence shown in SEQ ID NO: 1, preferably at least 75%, 80%, 85%, 90%, 92%, 94%, 96%, 98%, or 99% identical to SEQ ID NO: 1.

Preferably, the cell of the invention is mutated in at least one polynucleotide, where a subsequence having a size of at least 100 bp of the at least one polynucleotide hybridizes with a polynucleotide having the sequence shown in SEQ ID NO: 1, or the respective complementary sequence, under medium stringency hybridization conditions, preferably under medium-high stringency conditions, or more preferably under high stringency conditions.

In a preferred embodiment the mutated cell of the invention is one in which the gene encoding the polypeptide comprising an amino acid sequence at least 70% identical to the sequence shown in SEQ ID NO: 2, preferably at least 75%, 80%, 85%, 90%, 92%, 94%, 96%, 98%, or 99% identical to SEQ ID NO: 2, is partially or fully deleted from the chromosome; or comprises at least one frameshift mutation or non-sense mutation.

A preferred result of these mutations is, that the cell of the invention has at least a two-fold reduced expression-level of a polypeptide comprising an amino acid sequence at least 70% identical to the sequence shown in SEQ ID NO: 2, preferably at least 75%, 80%, 85%, 90%, 92%, 94%, 96%, 98%, or 99% identical to SEQ ID NO: 2; when compared with the otherwise isogenic but non-mutated cell; or that the cell has no measureable expression of said polypeptide, when compared with the otherwise isogenic but non-mutated cell.

### Polypeptide of Interest

In a preferred embodiment, the polypeptide of interest may be obtained from a bacterial or a fungal source.

For example, the polypeptide of interest may be obtained from a Gram positive bacterium such as a Bacillus strain, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis; or a Streptomyces strain, e.g., Streptomyces lividans or Streptomyces murinus; or from a Gram negative bacterium, e.g., E. coli or Pseudomonas sp.

The polypeptide of interest may be obtained from a fungal source, e.g. from a yeast strain such as a Candida, Kluyveromyces, Pichia, Saccharomyces, Schizosaccharomyces, or Yarrowia strain, e.g., Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis strain.

The polypeptide of interest may be obtained from a filamentous fungal strain such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain, in particular the polypeptide of interest may be obtained from an Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride strain.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

For purposes of the present invention, the term “obtained from” as used herein in connection with a given source shall mean that the polypeptide of interest is produced by the source or by a cell in which a gene from the source has been inserted.

The polypeptide of interest may be a peptide or a protein. A preferred peptide according to this invention contains from 2 to 100 amino acids; preferably from 10 to 80 amino acids; more preferably from 15 to 60 amino acids; even more preferably from 15 to 40 amino acids.

In a preferred embodiment, the protein is an enzyme, in particular a hydrolase (class EC 3 according to Enzyme Nomenclature; Recommendations of the Nomenclature Committee of the International Union of Biochemistry). In a particular preferred embodiment the following hydrolases are preferred:

### Proteases

Suitable proteases include those of animal, vegetable or microbial origin. Microbial origin is preferred. Chemically modified or protein engineered mutants are included. The protease may be an acid protease, a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235 and 274.

Preferred commercially available protease enzymes include ALCALASE™, SAVINASE™, PRIMASE™, DURALASE™, ESPERASE™, RELASE™ and KANNASE™ (Novozymes A/S), MAXATASE™, MAXACAL™, MAXAPEM™, PROPERASE™, PURAFECT™, PURAFECT OXP™, FN2™, and FN3™ (Genencor International Inc.).

### Lipases

Suitable lipases include those of bacterial or fungal origin. Chemically modified or protein engineered mutants are included. Examples of useful lipases include lipases from Humicola (synonym Thermomyces), e.g. from H. lanuginosa (T. lanuginosus) as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include LIPOLASE™, LIPOLASE ULTRA™ and LIPEX™ (Novozymes A/S).

### Amylases

Suitable amylases (alpha and/or beta) include those of bacterial or fungal origin. Chemically modified or protein engineered mutants are included. Amylases include, for example, alpha-amylases obtained from Bacillus, e.g. a special strain of B. licheniformis, described in more detail in GB 1,296,839.

Examples of useful amylases are the variants described in WO 94/02597, WO 94/18314, WO 96/23873, WO 97/43424, and WO 01/66712, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are DURAMYL™, TERMAMYL™, FUNGAMYL™, NATALASE™, TERMAMYL LC™, TERMAMYL SC™, LIQUIZYME-X™ and BAN™ (Novozymes A/S), RAPIDASE™ and PURASTAR™ (from Genencor International Inc.).

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include CELLUZYME™, CAREZYME™, and CAREZYME CORE™ (Novozymes A/S), CLAZINASE™, and PURADAX HA™ (Genencor International Inc.), and KAC-500(B)™ (Kao Corporation).

### Oxidoreductases

Oxidoreductases that may be treated according to the invention include peroxidases, and oxidases such as laccases, and catalases.

Other preferred hydrolases are carbohydrolases including MAN NAWAY™. Other preferred enzymes are transferases, lyases, isomerases, and ligases.

### Expression Constructs for the Polypeptide of Interest

In a preferred embodiment the cell of the invention comprises one or more chromosomally integrated copies of a polynucleotide encoding the at least one heterologous polypeptide.

It is preferred that the at least one heterologous polypeptide of the invention is encoded by a polynucleotide which is transcribed from at least one heterologous promoter; preferably the at least one promoter comprises an artificial promoter. Suitable promoter constructs are disclosed in WO 93/10249 which is incorporated herein in its entirety by reference.

In addition, the preferred artificial promoter comprises one or more mRNA-stabilizing sequence, preferably derived from the cryIIIa promoter. Suitable constructs are described in WO 99/43835 which is incorporated herein in its entirety by reference.

### Fermentations

The present invention may be useful for any fermentation in industrial scale, e.g. for any fermentation having culture media of at least 50 litres, preferably at least 100 litres, more preferably at least 500 litres, even more preferably at least 1000 litres, in particular at least 5000 litres.

The bacterial strain or cell may be fermented by any method known in the art. The fermentation medium may be a complex medium comprising complex nitrogen and/or carbon sources, such as soybean meal, soy protein, soy protein hydrolysate, cotton seed meal, corn steep liquor, yeast extract, casein, casein hydrolysate, potato protein, potato protein hydrolysate, molasses, and the like. The fermentation medium may be a chemically defined media, e.g. as defined in WO 98/37179.

The fermentation may be performed as a batch, a fed-batch, a repeated fed-batch or a continuous fermentation process.

In a fed-batch process, either none or part of the compounds comprising one or more of the structural and/or catalytic elements is added to the medium before the start of the fermentation and either all or the remaining part, respectively, of the compounds comprising one or more of the structural and/or catalytic elements is fed during the fermentation process. The compounds which are selected for feeding can be fed together or separate from each other to the fermentation process.

In a repeated fed-batch or a continuous fermentation process, the complete start medium is additionally fed during fermentation. The start medium can be fed together with or separate from the structural element feed(s). In a repeated fed-batch process, part of the fermentation broth comprising the biomass is removed at time intervals, whereas in a continuous process, the removal of part of the fermentation broth occurs continuously. The fermentation process is thereby replenished with a portion of fresh medium corresponding to the amount of withdrawn fermentation broth.

In a preferred embodiment of the invention, a fed-batch, a repeated fed-batch process or a continuous fermentation process is preferred.

### Recovery of the Polypeptide of Interest

A further aspect of the invention concerns the downstream processing of the fermentation broth. After the fermentation process is ended, the polypeptide of interest may be recovered from the fermentation broth, using standard technology developed for the polypeptide of interest. The relevant downstream processing technology to be applied depends on the nature of the polypeptide of interest.

A process for the recovery of a polypeptide of interest from a fermentation broth will typically (but is not limited to) involve some or all of the following steps:

1) pre-treatment of broth (e.g. flocculation)

2) removal of cells and other solid material from broth (primary separation)

3) filtration

4) concentration

5) filtration

6) stabilization and standardization.

Apart from the unit operations listed above, a number of other recovery procedures and steps may be applied, e.g., pH-adjustments, variation in temperature, crystallization, treatment of the solution comprising the polypeptide of interest with active carbon, and use of various adsorbents.

By using the method of the invention the yield of the polypeptide of interest is much higher in the recovery when the crystal formation is reduced or eliminated by adding of, e.g. MPG, during fermentation.

The invention is further illustrated in the following examples, which are not intended to be in any way limiting to the scope of the invention as claimed.

### EXAMPLES

### Media

LB agar, TY buillon medium and BPX shake flask medium have all been described in WO 94/14968. PS-1 shake flask medium (10% sucrose, 4% soybean flour, 1% Na₂SO₄. 12H₂O, 0.5% I CaCO₃, and 0.01% pluronic acid) has been described in Example 28 of U.S. Pat. No. 6,255,076.

### Strains and Donor Organisms

Bacillus subtilis PL1801

This strain is the B. subtilis DN1885 with disrupted apr and npr genes (Diderichsen, B., Wedsted, U., Hedegaard, L., Jensen, B. R., Sjoholm, C. (1990) Cloning of aldB, which encodes alpha-acetolactate decarboxylase, an exoenzyme from i Bacillus brevis. J. Bacteriol., 172, 4315-4321).

Bacillus licheniformis PP1962

This strain is a derivative of strain MDT223 disclosed in WO 2005/123915 (Novozymes), with the following additional modifications, described in three steps:

Step 1: The B. licheniformis L-aminopeptidase gene was inserted as to replace the protease gene present at the amyL locus in MDT223. An integration vector carrying the L-aminopeptidase gene from B. licheniformis flanked by the heterologous tandem/cryIIIa promoter 5′ region upstream of the protease gene and the 3′ amyL region was introduced by conjugation, and integrated into and excised from the chromosome as described in WO 1996/029418 (Novozymes).

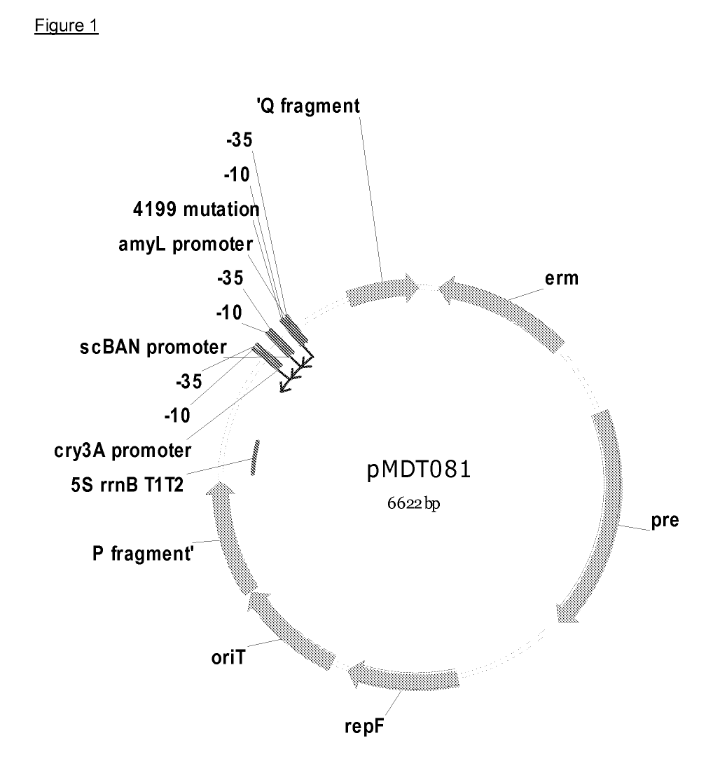

Step 2: A tandem/cryIIIA promoter, disclosed in WO 1999/043835 (Novozymes), followed by the E. coli rrnB transcriptional terminator, was inserted at the gntP locus, thereby creating a gntP deletion. The plasmid pMDT081 (SEQ ID NO:3; FIG. 1) was used for integration at the gntP locus on the chromosome of B. licheniformis.

Step 3: The ribosome binding site (RBS) of the L12 gene was modified to provide a strong reduction in L12 protein expression. An integration vector plasmid with the variant ribosome binding site was introduced and the variant gene was inserted into the chromosome, replacing the native L12 gene, by integration and excision as described in WO 1996/029418 (Novozymes). A resulting erythromycin-sensitive strain, containing the variant L12 gene was isolated. The final change of the L12 RBS was as follows (the underlined bases are the start codon of the L12 gene):

Wild type sequence:
(SEQ ID NO: 4)
atgaaagaggaggaatgaaataatg
The mutated EF variant:
(SEQ ID NO: 5)
atgaaagacgcgtaatgaaataatg

B. licheniformis PL4198

This strain is a derivative of strain MDT223 described in WO 2005/123915 with the following stepwise additional modifications:

Step 1: A tandem/cryIIIA promoter, disclosed in WO 1999/043835 (Novozymes), followed by the E. coli rrnB transcriptional terminator, was inserted at the gntP locus, thereby creating a gntP deletion. The plasmid pMDT081 (SEQ ID NO: 3) was used for integration at the gntP locus on the chromosome of B. licheniformis.

Step 2: The ribosome binding site (RBS) of the L12 gene was modified to provide a strong reduction in L12 protein expression. An integration vector plasmid with the variant ribosome binding site was introduced and the variant gene inserted into the chromosome, replacing the native L12 gene, by integration and excision as described above. A resulting erythromycin-sensitive strain, containing the variant L12 gene was isolated. The final change of the L12 RBS was as already shown in step 1) for the PP1962 strain.

Step 3: The B. licheniformis amyL gene was inserted as to replace the JP170 protease gene present at the amyL locus in MDT223. An integration vector plasmid carrying the amyL gene from B. licheniformis flanked by the heterologous tandem/cryIIIA promoter 5′ region upstream of the protease gene and the 3′ amyL region was introduced by conjugation, and integrated into and excised from the chromosome as described in WO 1996/029418 (Novozymes).

Step 4: A modified B. subtilis aprE protease gene (SEQ ID NO: 6) was inserted to replace the amyL gene inserted in step 3. An integration vector plasmid carrying the protease gene flanked by the 5′ tandem/cryIIIA and amyL 3′-segments was introduced by conjugation, and integrated into and excised from the chromosome as described above.

Step 5: the spoIIAC gene (sigF) was inactivated by deletion of nucleotides 70 to 436 of the 765 bp spoIIAC gene. The deletion was carried out by standard procedures using temperature sensitive plasmids and homologous recombination.

Step 6: the pgsB-, pgsC-, and pgsAA-, genes were inactivated by deletion of a chromosomal region from nucleotide 607 in the pgsB gene to nucleotide 180 in the pgsAA gene (both nucleotides included). The deletion was carried out by standard procedures using temperature sensitive plasmids and homologous recombination.

Plasmid pAN829

A deleted version of the gene encoding the B. licheniformis putative metallopeptidase BL00829 was constructed by PCR using splicing by overlap extension (SOE) (Horton et al, 1989, Gene 77(1):61-68). The 5′ and 3′ regions of the BL00829 gene were PCR amplified from B. licheniformis SJ1904 DNA using primer AN354 (which introduced a 5′ sacII restriction site) and primer AN355 for the 5′ BL00829 fragment, and primers AN356 and AN357 (which introduced a NotI restriction site) for the BL00829 3′ fragment. The primer sequences are shown below:

Primer AN354 (SEQ ID NO: 7):
ttgcacccgcggatacgagggagtggcgatgt
Primer AN355 (SEQ ID NO: 8):
gaatgaataaaagtgaagcccaagagaaggctttttcacaagatta
Primer AN356 (SEQ ID NO: 9):
taatcttgtgaaaaagccttctcttgggcttcacttttattcattc
Primer AN357 (SEQ ID NO: 10):
aagcatgcggccgcgatctttctgcatcatatgc

PCR amplifications were run under standard conditions and the products with expected sizes 639 bp (5′ fragment) and 648 bp (3′ fragment) were visualized using a 1% agarose-0.5×TBE gel. The final SOE fragment was named 829Δ14 (SEQ ID NO:11) and was generated using primer pair AN354 and AN357 according to Horton et al, 1989. The truncated version of the BL00829 gene present on the SOE product 829Δ14, encode a polypeptide of 14 aa which is deleted in the middle 138 aa of the native BL00829 protein. Plasmid pAN829 (SEQ ID NO:12; FIG. 2) was constructed by ligating the PCR product 829Δ14, cut with restriction enzymes sacII and NotI, to a vector plasmid, which contains the temperature sensitive origin of pE194. This plasmid was used for deletion of the BL00829 gene from the chromosome of Bacillus licheniformis by a double cross-over event. Further description of suitable host strains and integration procedures may be found in WO 2005/123915.

### Example 1

### Construction of a Mutated B. licheniformis Alpha-Amylase Host

This example describes the construction of a B. licheniformis strain comprising two copies of a gene encoding a secreted alpha-amylase. A mutant of this strain was then constructed by introducing a deletion in the gene encoding the putative metalloprotease BL00829.

The alpha-amylase used in this example is the JE1 alpha-amylase polypeptide originally produced by an alkalophilic Bacillus sp. JP170. This is the gene contained on plasmid pTVB115, described in WO99/23211. This particular gene will in the following be referred to as “je1”.

### MOL2650: Two Copy Let Alpha-Amylase Strain

The jet gene was transferred to an integration vector designed to allow integration of the alpha-amylase expression cassette into the chromosome of a B. licheniformis strain, that already contains an artificial tandem/cryIIIA promoter integrated at the amyL locus and the xylA locus, as described in example 6 of WO2005/123915. This was done by using double homologous recombination in the cryIIIA stabilizer region of the promoter and in the downstream segments for amyL and xylA, respectively. Further description of suitable host strains and integration procedures may be found in WO 2005/123915.

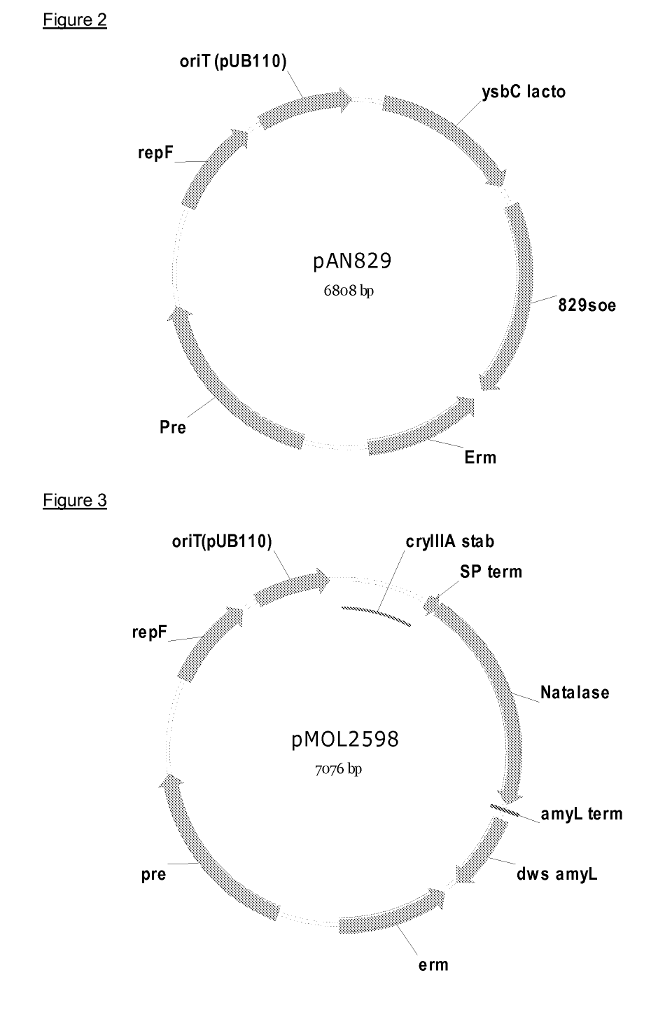

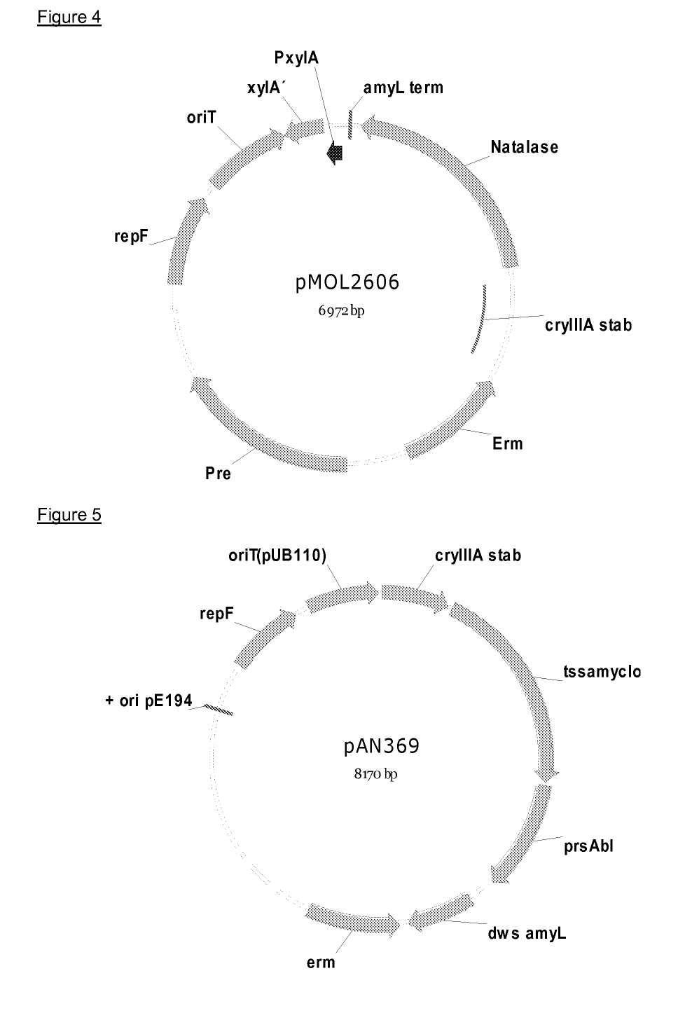

The integration vectors pMOL2598 (SEQ ID NO:13; FIG. 3) and pMOL2606 (SEQ ID NO:14; FIG. 4) for the amyL and xylA locus, respectively, were constructed by inserting the DNA fragment encoding JE1 amylase into a pE194 derivative vector. These plasmids can also be made by cloning PCR fragments from known standard vectors such as pE194 or ligating synthetic DNA fragments.

The vector pMOL2598 was transformed into B. subtilis PL1801 competent cells, selecting for erythromycin resistance (2 microgram/ml) at 30° C. A resulting transformant was MOL2598 (PL1801/pMOL2598). This plasmid was then re-transformed by either competence, electroporation or conjugation into a Bacillus licheniformis PP1962 described above, and by double homologous recombination the L-aminopeptidase gene was replaced with the je1 amylase gene. The resulting strain was isolated as MOL2614.

The vector pMOL2606 was also transformed into B. subtilis PL1801 competent cells, selecting erythromycin resistance (2 microgram/ml) at 30° C. A resulting transformant was MOL2608 (PL1801/pMOL2606). This plasmid was then re-transformed by either competence, electroporation or conjugation into a Bacillus licheniformis MOL2614 described above, and by double homologous recombination the je1 gene was integrated into the xyl locus. The resulting strain was isolated as MOL2650.

The final MOL2650 strain has two copies of the je1 amylase gene expressed from the strong promoter P17 described in WO2005/123915 example 6. The integration vector also contains the prsA gene from B. licheniformis in a position that places the cloned bmy1 gene upstream of prsA in a dicistronic operon. One of the copies is located in the amyL locus and a second copy is located in the xyl locus. The strain is furthermore deleted in the genes encoding the proteases AprE and C-component as described in patent WO2005/123915. Furthermore, the ribosome binding site of the L12 gene was modified, so as to lead to a strong reduction in L12 protein expression (see above).

### AN407: Mutated MOL2650 Strain

The plasmid pAN829 was transferred to Bacillus licheniformis MOL2650 (described above) by conjugation, and the BL00829 gene was replaced with the truncated BL00829 gene, t829, by double homologous recombination. The resulting strain was isolated as AN407

The AN407 strain is isogenic with the MOL2650 strain, except the gene encoding the putative metalloprotease BL00829 has been mutated by deletion.

### Example 2

### Construction of a Mutated B. licheniformis Beta-Amylase Host

This example describes the construction of two B. licheniformis strains comprising a gene encoding a secreted beta-amylase; the two strains were isogenic except one was mutated by a deletion in the gene encoding the putative metalloprotease BL00829. The beta-amylase used in this example was the beta-amylase originally produced by the thermophilic bacterium Clostridium thermosulfurogenes with a heterologous secretion signal sequence derived from B. licheniformis amyL instead of the native signal sequence. This particular hybrid gene will in the following be referred to as “bmy1” and its nucleotide sequence is shown in SEQ ID NO:15.

AN411: Two Copy bmy1 Beta-Amylase Strain

The bmy1 gene (SEQ ID NO:15) was transferred to an integration vector designed to allow integration of the beta-amylase expression cassette into the chromosome of a B. licheniformis strain, that already contains an artificial tandem/cryIIIA promoter integrated at the amyL locus and xylA locus, as described in example 6 of WO2005/123915. This was done by using double homologous recombination in the cryIIIA stabilizer region and in the downstream segments for amyL and xylA respectively. Further description of alternative suitable host strains and integration procedures may be found in WO 2005/123915.

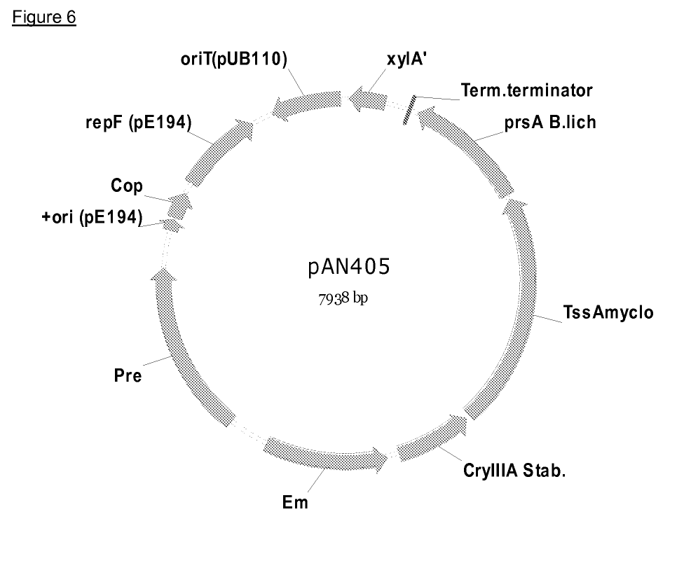

The integration vectors pAN369 (SEQ ID NO:16; FIG. 5) and pAN405 (SEQ ID NO:17; FIG. 6) for the amyL and xylA locus, respectively, were constructed by inserting the DNA fragment encoding bmy1 beta-amylase into a pE194 derivative vector. These plasmids could also be made by cloning PCR fragments from known standard vectors such as pE194 or ligating synthetic DNA fragments.

The vector pAN369 was transformed into B. subtilis PL1801 competent cells, selecting for erythromycin resistance (2 microgram/ml) at 30° C. A resulting transformant was AN369 (PL1801/pAN369). This plasmid was then re-transformed by either competence, electroporation or conjugation into the Bacillus licheniformis PL4198 strain described above, and by double homologous recombination the protease gene was replaced with the bmy1 beta-amylase gene. The resulting strain was isolated as AN374.

The vector pAN405 was also transformed into B. subtilis PL1801 competent cells, selecting erythromycin resistance (2 microgram/ml) at 30° C. A resulting transformant was AN405 (PL1801/pAN405). This plasmid was then re-transformed by either competence, electroporation or conjugation into a Bacillus licheniformis AN374 described above, and by double homologous recombination the bmy1 gene was integrated into the xyl locus. The resulting strain was isolated as AN411.

The final AN411 strain has two copies of the bmy1 beta-amylase gene expressed from the strong promoter P17 described in WO2005/123915 example 6. One of the copies is located in the amyL locus and a second copy is located in the xyl locus. The strain is furthermore deleted in the genes encoding the proteases AprE and C-component as described in patent WO2005/123915. Furthermore, the ribosome binding site of the L12 gene was modified, so as to lead to a strong reduction in L12 protein expression (see above).

### AN420: Mutated AN411 Strain

The plasmid pAN829 was transferred to Bacillus licheniformis AN411 (described above) by conjugation, and the putative metalloprotease encoding gene of SEQ ID NO:1 was replaced with the truncated version, t829, by double homologous recombination. The resulting strain was isolated as AN420.

The AN420 strain is isogenic with the AN411 strain, except the gene encoding the putative metalloprotease BL00829 has been mutated by deletion.

### Example 3

### Beta-Amylase Production in B. licheniformis Strains from Example 2

A fed-batch fermentation process of the Bacillus licheniformis strains from Example 2 was conducted as described below. All media were sterilized by methods known in the art. Unless otherwise described, tap water was used. The ingredient concentrations referred to in the below recipes are before any inoculation.

### Media

LB agar: 10 g/l peptone from casein; 5 g/l yeast extract; 10 g/l sodium chloride; 12 g/l Bacto-agar adjusted to pH 7.0+/−0.2. Premix from Merck was used (LB-agar (Miller) 110283).

M-9 buffer: Di-Sodiumhydrogenphosphate, 2H₂O 8.8 g/l; potassiumdihydrogenphosphate 3 g/l; sodium chloride 4 g/l; magnesium sulphate, 7H₂O 0.2 g/l (deionized water is used in this buffer).

PRK-50: 110 g/l soy grits; Di-sodiumhydrogenphosphate, 2H₂O 5 g/l; Antifoam (Struktol SB2121; Schill & Seilacher, Hamburg, Germany) 1 ml/l. pH adjusted to 8.0 with NaOH/H₃PO₄ before sterilization.

Make-up medium: Tryptone (Casein hydrolysate from Difco (Bacto™ Tryptone pancreatic Digest of Casein 211699) 30 g/l; magnesium sulphate, 7H₂O 4 g/l; di-potassiumhydrogenphosphate 7 g/l; di-sodiumhydrogenphosphate, 2H₂O 7 g/l; di-ammonium-sulphate 4 g/l; citric acid 0.78 g/l; vitamins (thiamin-dichlorid 34.2 mg/l; riboflavin 2.9 mg/l; nicotinic acid 23 mg/l; calcium D-pantothenate 28.5 mg/l; pyridoxal-HCl 5.7 mg/l; D-biotin 1.1 mg/l; folic acid 2.9 mg/l); trace metals (MnSO₄, H₂O 39.2 mg/l; FeSO₄, 7H₂O 157 mg/l; CuSO₄, 5H₂O 15.6 mg/l; ZnCl₂ 15.6 mg/l); Antifoam (Struktol SB2121; Schill & Seilacher, Hamburg, Germany) 1.25 ml/l; pH adjusted to 6.0 with NaOH/H₃PO₄ before sterilization.

Feed-medium: Glucose, 1H₂O 820 g/l

### Fermentation Procedure:

Bacillus licheniformis strains was grown on LB agar slants for one day at 37° C. The agar was then washed with M-9 buffer, and the optical density (OD) at 650 nm of the resulting cell suspension was measured. Inoculum shake flasks (with 100 ml medium PRK-50) were inoculated with an inoculum of OD (650 nm)×ml cell suspension=0.1. The shake flasks were incubated at 37° C. at 300 rpm for 20 hr.

The fermentors used were standard lab fermentors equipped with a temperature control system, pH control with ammonia water and phosphoric acid, dissolved oxygen electrode to measure >20% oxygen saturation through the entire fermentation.

The fermentation in the main fermentor (fermentation tank) was started by inoculating the main fermentor with the growing culture from a shake flask. The inoculated volume was 10% (80 ml for 720 ml make-up media, resulting in 800 ml initial broth after inoculation).

The fermentation parameters were: Temperature 38° C.; pH between 6.8 and 7.2 (using ammonia water and phosphoric acid, control 6.8 (ammonia water), 7.2 phosphoric acid). Aeration: 1.5 liter/min, agitation: 1500 rpm.

Feed-medium was added as follows: Initial feed rate 0.05 g/min/kg at the start of the fermentation, increasing linear to 0.16 g/min/kg after 8 hours and remaining at 0.16 g/min/kg until the end of fermentation, by reference to the starting weight of the fermentation broth, just after the inoculation. The fermentation was terminated after 3 days (approx. 70 hours).

### Beta-Amylase Assay

Beta-Amylase acts on the non-reducing end of maltohexaose (G6) to form maltose (G2) and maltotetraose (G4). Produced G4 reacts stronger than G6 in the presence of lactose-oxidase and O₂ to form H₂O₂. The formed H₂O₂ activates in the presence of peroxidase the oxidative condensation of 4-aminoantipyrine (AA) and N-ethyl-N-sulfopropyl-m-toluidine (TOPS), to form a purple product which can be quantified by its absorbance at 540 nm. The reaction is initiated by maltohexaose (G6). When all components but beta-amylase are in surplus, the rate of the rising absorbance is proportional to the beta-amylase activity present. The analysis is performed automatically by Konelab.

A beta-amylase unit (BAMU) is defined as the amount of enzyme that degrades one μmol maltohexaose per minute under the conditions described in this document. The activity is determined relative to an enzyme standard.

Sulfite and Termamyl don't interfere significantly with the BAMU results. Other hydrogen peroxide producing or consuming agents may show false BAMU activity.

### Equipment:

Konelab Arena® 30 Analyser

Analytic balance (e.g. Mettler AT200, Mettler AE100)

Dilution equipment (e.g. Hamilton diluter)

Magnetic Stirrer

pH meter (e.g. Radiometer PHM 93)

Pipettes

TABLE 1
Reaction conditions
Reaction conditions

| Buffer | 67 mM phosphate | |
|---|---|---|
| | and 67 mM citrate | |
| pH | 5.5 | |
| β-Amylase | 0.083-0.166 | BAMU/mL |
| Maltohexaose | 0.856 | mM |
| Lactose oxidase | 4.8 | LOXU/mL |
| 4-Aminoantipyrine (AA) | 1.7 | mM |
| N-Ethyl-N-sulfopropyl-m-toluidine (TOPS) | 4.3 | mM |
| Peroxidase (Sigma) | 2.1 | U/mL |
| Temperature | 37° | C. |
| Reaction time | 200 | sec. |
| Wavelength | 540 | nm |

TABLE 2
Specificity and sensitivity.

| | Minimum dilution | Quantification limit, BAMU/g |
|---|---|---|
| Solid samples | 1 g up to 25 mL | 2.075 BAMU/g |
| Liquid samples | 1 g up to 25 mL | 2.075 BAMU/g |

Results of samples with activity lower than the quantification limit are reported as <2.075 BAMU/g.
Quantification range (0.083-0.166) BAMU/mL

TABLE 3
Diluent, Brij 85 ppm, Calcium 30 mM.

| Step | Action |
|---|---|
| 1 | Weigh out 4.41 ± 0.02 g CaCl₂•H₂O (e.g. |
| | Merck 1.02382) and transfer to a 1000 mL |
| | volumetric flask. |
| 2 | Add 85 ± 5 μL BRIJ 35, 30% solution (e.g. |
| | Sigma S430AG6). |
| 3 | Add approximately 800 mL deionised water. |
| 4 | Mix on a magnetic stirrer until fully dissolved. |
| 5 | Make up to the mark by deionised water. |

Stability: 2 weeks at room temperature.

TABLE 4
Buffer, phosphate 100 mM, citrate 100 mM, pH 5.5.

| Step | Action |
|---|---|
| 1 | Weigh out 17.80 ± 0.05 g Na₂HPO₄•2H₂0 |
| | (e.g. Merck 6580) and 21.00 ± 0.05 g citrate |
| | C₆H₈O₇ (e.g. Merck 244), and transfer to a |
| | 1000 mL volumetric flask. |
| 2 | Add approximately 800 mL deionised water. |
| 3 | Mix on a magnetic stirrer until fully dissolved. |
| 4 | Adjust pH to 5.50 ± 0.05 by using NaOH. |
| 5 | Make up to the mark by deionised water. |

Stability: For immediate use.

TABLE 5
BAMU Reagent, TOPS 10 mM, AA 4 mM, 4.8 LOXU/mL.

| Step | Action |
|---|---|
| 1 | Weigh out and transfer into a 50 mL |
| | volumetric flask: 150 ± 2 mg TOPS (N-ethyl- |
| | N-sulfopropyl-m-toluidine•H₂O, e.g. Sigma E- |
| | 8506, 297 g/mol), 41 ± 1 mg AA (4- |
| | aminoantipyrine, e.g. Sigma A-4382), 3 ± 0.5 |
| | mg Peroxidase (e.g. Sigma P-8125, 96 |
| | U/mg), 200 ± 2 mg LOXU standard. |
| 2 | Fill up to the mark with cold (1-6° C.) citrate |
| | phosphate buffer. |
| 3 | Mix on a magnetic stirrer without heating until |
| | totally dissolved. |
| 4 | Make 5.5 mL aliquots and store them frozen |
| | and protected from light, e.g. by wrapping in |
| | aluminium foil. |

Stability: 30 days in a freezer protected from light, or until purple colour is visible after thawing.

TABLE 6
Maltohexaose substrate 3.668 mM.

| Step | Action |
|---|---|
| 1 | Weigh out 95 ± 1 mg maltohexaose (e.g. |
| | Sigma M-9153) and transfer to a 25 mL |
| | volumetric flask. |
| 2 | Add citrate phosphate buffer up to the mark. |
| 3 | Mix on a magnetic stirrer until totally |
| | dissolved. |

Stability: 25 days refrigerated.

TABLE 7
Standard stock solution, 1.66 BAMU/mL.

| Step | Action |
|---|---|
| 1 | Weigh out 0.7477 ± 0.0005 g of the standard |
| | (222 BAMU/g) and transfer to a 100 mL |
| | volumetric flask. |
| 2 | Fill up to the mark with diluent. |
| 3 | Mix on a magnetic stirrer for 15 minutes. |

TABLE 8
Standard dilutions; stable for 4 hours at room temperature.

| Dil. No. | Action | BAMU/mL |
|---|---|---|
| 1 | 20 | 0.083 |
| 2 | 16 | 0.104 |
| 3 | 13 | 0.128 |
| 4 | 11 | 0.151 |
| 5 | 10 | 0.166 |

TABLE 9
Level control: The level control dilution
is stable for 4 hours at room temperature.

| Step | Action |
|---|---|
| 1 | Weigh out 0.5 g of the current level control |
| | Raizyme R0401013WBA (1281 BAMU/g) |
| | and transfer into a 250 mL volumetric flask. |
| 2 | Fill up to the mark with diluent. |
| 3 | Mix on a magnetic stirrer for 15 minutes. |
| 4 | Dilute the solution further by factor x20 with |
| | Hamilton Diluter directly into sample cup. |

The solutions should be stirred before use.

TABLE 10
Sample dilutions: Stable for 4 hours at room temperature.

| Step | Action |
|---|---|
| 1 | Weigh out and dissolve the samples in |
| | diluent in a volumetric flask. |
| 2 | Mix on a magnetic stirrer for 15 minutes. |
| 3 | Dilute further with diluent to reach 0.0915 |
| | BAMU/mL target concentration. |

The solutions should be stirred before use

Prepare reagents, standards, level control and sample dilutions as described in previous sections. Place the reagents in the Konelab analyser. Use “Maltohexaose substrate 3.668 mM” as BAMU-SUB and use “BAMU Reagent, TOPS 10 mM, AA 4 mM, 4.8 LOXU/mL” as BAMU-AAT (table 12).

TABLE 11
Reagents.

| Reagent | Insert reagent | Vessel size | Pipette Mode |
|---|---|---|---|
| BAMU Reagent, TOPS | BAMU-AAT | 60 mL | Normal |
| 10 mM, AA 4 mM, | | | |
| 4.8 LOXU/mL | | | |
| Maltohexaose substrate | BAMU-SUB | 60 mL | Normal |
| 3.668 mM | | | |

Place the samples in the Konelab and analyse all sample solutions in the order as follows (max. 28 samples per run):

1. Blank

2. Standard 1

3. Standard 2

4. Standard 3

5. Standard 4

6. Standard 5

7. Level control

8. Sample

9. . . .

n. Sample

Calculate the standard curve by linear regression of Abs versus concentration or use AnaAdm. Calculate activities in BAMU/mL for the level control and the samples or use AnaAdm. Correct the activities in BAMU/mL for dilution and weighing or use AnaAdm. Example: 1.0000 g sample is dissolved in a 100 mL volumetric flask and diluted further 10 times. The result calculated from the standard curve is 0.140 BAMU/mL. The activity in the sample is: 0.140 BAMU/mL*100 mL*10/1.0000 g=140 BAMU/g.

The intermediate precision for the BAMU assay is:

CV % for a single determination=5.04%

CV % approval for BAMU was calculated to be 9.1%

### Beta-Amylase Results:

TABLE 12
Relative yields of bmy1 beta-amylase produced in
B. licheniformis strains AN411 and AN420 (see
example 2) in lab-scale fed-batch cultivations:

| Time (hours) | AN411 | AN420 |
|---|---|---|
| 25.73 | 100 | 144 |
| 52.65 | 100 | 144 |
| 67.33 | 100 | 165 |

1.-39. (canceled)

40. A mutated Bacillus cell producing at least one heterologous polypeptide of interest, wherein said cell has a reduced expression-level of a metallopeptidase an amino acid sequence at least 70% identical to the sequence shown in SEQ ID NO: 2, when compared with an otherwise isogenic but non-mutated cell.

41. The cell of claim 40 which is a Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, or Bacillus thuringiensis cell.

42. The cell of claim 40, which is mutated in a gene encoding the metallopeptidase, wherein the gene comprises a polynucleotide having a nucleotide sequence at least 70% identical to the sequence shown in SEQ ID NO: 1.

43. The cell of claim 42, in which the gene is partially or fully deleted from the chromosome or comprises at least one frameshift mutation or at least one non-sense mutation.

44. The cell of claim 40, which has no measureable expression of the metallopeptidase, when compared with the otherwise isogenic but non-mutated cell.

45. The cell of claim 40, wherein the at least one heterologous polypeptide comprises an enzyme.

46. The cell of claim 40, wherein the enzyme is a lyase, ligase, a hydrolase, an oxidoreductase, a transferase, or an isomerase.

47. The cell of claim 40, which has an improved production of the heterologous polypeptide of interest, when compared with the otherwise isogenic but non-mutated cell.

48. A method for constructing a mutated Bacillus cell, said method comprising the steps of:
a) mutating a Bacillus cell; and
b) selecting a mutated cell which has a reduced expression-level of a metallopeptidase comprising an amino acid sequence at least 70% identical to the sequence shown in SEQ ID NO: 2, when compared with an otherwise isogenic but non-mutated cell.

49. The method of claim 48, wherein the Bacillus cell is a Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, or Bacillus thuringiensis cell.

50. The method of claim 48, wherein the Bacillus cell in step (a) is mutated in a gene encoding the metallopeptidase, wherein the gene comprises a polynucleotide having a nucleotide sequence at least 70% identical to the sequence shown in SEQ ID NO: 1.

51. The method of claim 50, wherein the Bacillus cell in step (a) is mutated by partial or full deletion of the gene from the chromosome of the cell or by introducing at least one frameshift mutation or at least one non-sense mutation in the gene.

52. A method for producing a heterologous polypeptide of interest, said method comprising the steps of:
a) cultivating a mutated Bacillus cell producing at least one heterologous polypeptide of interest, wherein said mutated Bacillus cell has a reduced expression-level of a metallopeptidase comprising an amino acid sequence at least 70% identical to the sequence shown in SEQ ID NO: 2, when compared with an otherwise isogenic but non-mutated cell, and
b) isolating the polypeptide of interest.

53. The method of claim 52, wherein the Bacillus cell is a Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, or Bacillus thuringiensis cell.

54. The method of claim 52, wherein the Bacillus cell in step (a) is mutated in a gene encoding the metallopeptidase, wherein the gene comprises a polynucleotide having a nucleotide sequence at least 70% identical to the sequence shown in SEQ ID NO: 1.

55. The method of claim 54, wherein the Bacillus cell in step (a) is mutated by partial or full deletion of the gene from the chromosome of the cell or by introducing at least one frameshift mutation or at least one non-sense mutation in the gene.

56. The method of claim 52, wherein the at least one polypeptide of interest comprises an enzyme.

57. The method of claim 56, wherein the enzymes is a lyase, a ligase, a hydrolase, an oxidoreductase, a transferase, or an isomerase.


SEQUENCE LISTING (17 sequences)

SEQ ID NO: 1 - DNA, 456 bp, Bacillus licheniformis
  CDS (1)..(453): Predicted open reading frame encoding a putative metalloprotease having the amino acid sequence shown in SEQ ID NO2.
atgaataaaagtgaagcccaacagtttttaagcaacatgtatcaggacatcgtgtcggaaatgaatactgaaaaaattgccgattatttcagcgaggattatgttcaagtcacggacggcagtcacattgaccttgtgcagtttaaagatcacatacggacgctgaaaaacgttgcacgcaccataacggtgtcgccctttcatgaatttttgtttgacgagcgcctcgaaagcgcagcagtcagatatacggtcaaggttgtcaaaaagaatggagaccgggggagaattgatatcatcgcgatttttaaaatgaacggttttaaaatcgttcaatgtcacgagctttcacacgcgagcgaaggtacgaagggtattgaagagctggcgaagatcagtgaggcagcaattgaaaaagcctcctcccgggaggagaaggctttttcacaagattaa

SEQ ID NO: 2 - protein, 151 aa, Bacillus licheniformis
MNKSEAQQFLSNMYQDIVSEMNTEKIADYFSEDYVQVTDGSHIDLVQFKDHIRTLKNVARTITVSPFHEFLFDERLESAAVRYTVKVVKKNGDRGRIDIIAIFKMNGFKIVQCHELSHASEGTKGIEELAKISEAAIEKASSREEKAFSQD

SEQ ID NO: 3 - DNA, 6622 bp, Artificial sequence
  Plasmid pMDT081
gcggccgcaaccatttgatcaaagcttgcatgcctgcaggtcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagccgcggatatcgattcgttcgatgtcgtctccgacatggtcggaagcacgcacagacacgcgccgaaagaagaatcggccaaagaatacagaaaattaatgccgatctttatcaacttatctcgagcactggaaaacgagtatacacaaattgcaaattatcaaagaagcttaaccagcaaaaaataggggaagaattggaggcgttatcatgccattattaatcgtagcaatcgggattgtcgcattattgcttttgattatggggttaaaactgaatacgtttgtttccttgattatcgtatcgttcggtgttgcattggcacttggaatgccgtttgacgacatcgtcaaaacgattgaacaaggccttggcggaacgctcggccatatcgcattgatcttcgggctcggcgccatgctgggcaagctgatcgcggattcagggggcgctcagcgcattgcgaagaccctcgtcaacaaattcggggagaaaaacatccaatgggcggtcgtcatcgcctcctttatcatcggggttgctttattcttcgaagtcggattggttttattgattccaattgtattcgcaatttcaagagaattgaagatttctattttatacctcggaattcgcccttgagctctaagtgactagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctgttgggaataatttatacactattctattggaatcttaatcattccaatagaaaaatatgtaatgattataaataagtcgcttcttatcataaatatatttacatattcatttaatactacatcatgttaggtatagtaaggctatcaagggtgtcttaatttctacttgtaacaatgtattggcatattatatattgaattgagaaaattaaatacagcgataattcacatgaacaagttcattggtagttatattttcaaattttcaaggttgtgcttgtatgtcattctatagttagataagcatttgaggtagagtccgtccgaatatatttgtaatctgaagaaggttcaaacatatttctatataacgtattctttttttgtagttcttacttttgaggggcgttacaattcaaagatattatctttaattaagcttaacattaataattcttcaattgcaacaaaaaaagcacttttatctaaggtttcatcttacgtttcgagggcccctccattttcttatacaaattatattatacatatcagtaaaataatgtcaacccccctttattccttttttttacacagcggacagtctggacagcaggcccctcctttcaatgtgatacatatgatattgtataaatattccgaatttttaacaagtaccattttccctatattttcttccaaaagaaaagcgccgatatggcgctttctactcatttattcaatagcctctctgcttcttcacttcttcaagctgagatacagttaccaattgatagccttttgctttcagctttttaataatctcttcagcagcatctgcggacgttgcataaatatcgtgcattaagacgatttttccgtctcccgcatggctcatgacatgattgacaatcttttgcttatttttgtacttccaatcttccggatcaacatcccacaatgaaaccttcagattggaaagcgagcggccgtccgaaaggcgcgggtcattttgtcaaaatggtgcacaacggcatcgaatacgcagacatgcagctcatcgccgaagcatatacgtttttaagagaaaagcttcttttgccgatagatgaaatcgctgacattttcgacacgtggaatcaaggagagctgaacagctatttaatcgaaatcacggcggagatcctgcggaaaaaggatgagaagacgggcgctccactcatcgacgtcatcctcgacaaaaccggccaaaaaggcacgggcaaatggacgagcctgcaggccgtcgacaacggcattccatcatcaattatcacggaatccctgtttgcccgttacctgtcatcattaaaagacgaacggaca

SEQ ID NO: 4 - DNA, 25 bp, Bacillus licheniformis
  RBS (1)..(25): Wildtype L12 RBS
atgaaagaggaggaatgaaataatg

SEQ ID NO: 5 - DNA, 25 bp, Artificial sequence
  Artificial RBS variant derived from SEQ ID NO4.
atgaaagacgcgtaatgaaataatg

SEQ ID NO: 6 - DNA, 1143 bp, Artificial sequence
  A modified B. subtilis aprE protease gene.
atgaagaaaccgttggggaaaattgtcgcaagcaccgcactactcatttctgttgcttttagttcatcgatcgcatcggctgctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacggcaatggcgcaatcggtaccatggggaattagccgtgtgcaagccccagctgcccataaccgtggattgacaggttctggtgtaaaagttgctgtcctcgatacagggatatccactcatccagatctaaatattcgtggtggcgcaagctttgtaccaggggaaccgtcgactcaagatgggaacgggcatgggacgcacgttgcaggaacgattgcggctcttgataattcaatcggtgtgattggtgtggcaccaagtgctgatctatacgctgtaaaagtacttggagcaaatggtagaggaagcgttagtggaattgctcaaggtctagagtgggctgcagcgaataacatgcatattgctaacatgagtctcggtagtgatgcacctagtactacacttgagcgtgcagtcaactacgcgacaagccaaggtgtactagttattgcagcgactggtaacaacggttctggttcagttggctatcctgctcgttatgcaaacgcaatggctgtaggagcgactgaccaaaacaacagacgtgcaaacttttctcagtacggtacaggaattgacatcgtagcacctggagttaacgtacaaagtacgtatccaggaaaccgttatgtgagtatgaatggtacatctatggccactccacacgtcgccggcgtcgccgcccttgttaaacaaaagaacccatcttggtctaatgtacaaattcgaaatcatctaaagaatacggcaactagtttaggaagcacgaacttgtatggaagcggacttgttaacgcagaagcggcaacgcgttaa

SEQ ID NO: 7 - DNA, 32 bp, Artificial sequence
  Primer AN354
ttgcacccgcggatacgagggagtggcgatgt

SEQ ID NO: 8 - DNA, 46 bp, Artificial sequence
  Primer AN355
gaatgaataaaagtgaagcccaagagaaggctttttcacaagatta

SEQ ID NO: 9 - DNA, 46 bp, Artificial sequence
  Primer AN356
taatcttgtgaaaaagccttctcttgggcttcacttttattcattc

SEQ ID NO: 10 - DNA, 34 bp, Artificial sequence
  Primer AN357
aagcatgcggccgcgatctttctgcatcatatgc

SEQ ID NO: 11 - DNA, 1247 bp, Artificial sequence
  SOE fragment named 829delta14 generated using primer pair AN354 and AN357.
aagcatgcggccgcgatctttctgcatcatatgcttttttttgcttgctttaattattgttaactggtaaactatttaatagttaaccggttaatgattgtgaaaaggaatgatgacagtggaaaacaagacgtatgagcaggcatttcaagccgttcaggattttattctcaaaagagaaaagcgggctcagcatgaacagcaggcgttcgcagaggaagcctttcaagaagaagaaagcatgcgtaagcattggaccctgacccagcttcacattatctcgcttatcagcgaaagcgaagcagacgtcaacaacgctttcctggcagcaaaactccaaatttcaaaagcggcagtgacaaaagcggtcaatgtgctcacaaagcacggaatgatcgaatcgcacaaaaaaccgaacaacaacaaagaattgtattacacattaaccgatgaggggaaaaaactggccgacattcatgacaggatgcatgaaattgcaaaacagcgctacatcgagctgtttgatcggttttccgaatccgagctgcaaaccgtcatccgctttttaaacgaatggtcaaaacacatttaatcaagaggagcagcgaatgaataaaagtgaagcccaagagaaggctttttcacaagattaatgtttggcatcaaaccggatgttgagcgagacgggtccgcgtgtgtaaagccctgtctcgcggtaaatgaagtcttcctccagccggatatttttcagctgatcaaggacgatatttgcgacaagctcgatttcggtcttcgcaaaccccgctcctacacagttgtggacgcctgaaccgaatgcgagatgcctggctgcgccgctgaatgcgcttttgacttccaggtcgctgcggtgaatgttgaacttgtcgggatcctcgaacgcttcaggatcgcgatttgccgcgcctatcatgcaaaatacagtcgtcccttcttttagctcgactccgccaatctccgcgtcttgtgaaagctggcgcgggatgagctgcaccggcggcttgtagcgcagcgtctctgcgattgcctgagggagaagcgtgcggtcttccagcacatcattcatctgatcgggatgatgaagcaaatgataaatcattaatgcgagcgtcttgtccgccggttctgtggcggcaagcaatatattgagaatcagcgcgcgtatgtcccggtcagacatcgccactccctcgtatccgcgggtgcaa

SEQ ID NO: 12 - DNA, 6808 bp, Artificial sequence
  Plasmid pAN829
aattcagatccttattgttcctgaaattggcaccgctaaaattaatgaagtgacttttgaagaaatgaaagactatctcaaaaaataacttgagttattctaagagaaaccctatctaaaaatcacaaaaatgtggtagcattaaaaaaatcccagcttttataaaagctgtaaaaacacttatttaaaggagaaacaatgtatatttacctagcttttgcattagttggcggttttttacttgctaatcaaaatccaatcaatgcggatttacgaaaaattgttggctcaccatttttggcctctggaatttccaactttgttggttcgatttttttaggaattatcactttagtgaccagtcaaacactttttcctagttttcaatttgttggctcacacccagtatggatatggattggtggggttcttggtgggatttttctaacatctaatgttttacttttcccaagattaggagctgtccaaacggtgattttacctattttgggtcgaatattgatggggacacttattgattcatttggctggtttcatgccatgcaacttccgatgactctgatgcgctttttgggagttatcattactttagctggggttattgtcgcggttgttcttcctaatttaaaagaaaaagaagcagaaacgcaccaaactaacttactaggctggcgaatttgggcggtcatcgttggggcaatgtcggctgctcaacaagcaattaatggcagattgggagttttacttgaaaacactgcacaagcaacctttgtttcgttcttcattggatttttagctatttttatcgtgtctctttttattgaccgccgtttgccaaaaatttcagaattaaaaaaagcaaaaccttggaatggaattggtggatttttaggagcctcaatcgtttttgcaacagtcgttgctgttccgcaaattggtgcagggctgacaattatgatgggcttgattggacaaattttaggcagtatgttggttcaacaatttggttggtggcgctcaagtaaatatggcattcaaatttggcaaattgttgggattctaattatgctgaccggaataatattcattaaatttttataatgtagctagtaatatggaacagcgtttcacgctcactaccttccgtctgcgcgttcaataagcagtaatacctataaaagaacttagaaaattgaggataatcactctaaaatgtttactcagatctccgcggatacgagggagtggcgatgtctgaccgggacatacgcgcgctgattctcaatatattgcttgccgccacagaaccggcggacaagacgctcgcattaatgatttatcatttgcttcatcatcccgatcagatgaatgatgtgctggaagaccgcacgcttctccctcaggcaatcgcagagacgctgcgctacaagccgccggtgcagctcatcccgcgccagctttcacaagacgcggagattggcggagtcgagctaaaagaagggacgactgtattttgcatgataggcgcggcaaatcgcgatcctgaagcgttcgaggatcccgacaagttcaacattcaccgcagcgacctggaagtcaaaagcgcattcagcggcgcagccaggcatctcgcattcggttcaggcgtccacaactgtgtaggagcggggtttgcgaagaccgaaatcgagcttgtcgcaaatatcgtccttgatcagctgaaaaatatccggctggaggaagacttcatttaccgcgagacagggctttacacacgcggacccgtctcgctcaacatccggtttgatgccaaacattaatcttgtgaaaaagccttctcttgggcttcacttttattcattcgctgctcctcttgattaaatgtgttttgaccattcgtttaaaaagcggatgacggtttgcagctcggattcggaaaaccgatcaaacagctcgatgtagcgctgttttgcaatttcatgcatcctgtcatgaatgtcggccagttttttcccctcatcggttaatgtgtaatacaattctttgttgttgttcggttttttgtgcgattcgatcattccgtgctttgtgagcacattgaccgcttttgtcactgccgcttttgaaatttggagttttgctgccaggaaagcgttgttgacgtctgcttcgctttcgctgataagcgagataatgtgaagctgggtcagggtccaatgcttacgcatgctttcttcttcttgaaaggcttcctctgcgaacgcctgctgttcatgctgagcccgcttttctcttttgagaataaaatcctgaacggcttgaaatgcctgctcatacgtcttgttttccactgtcatcattccttttcacaatcattaaccggttaactattaaatagtttaccagttaacaataattaaagcaagcaaaaaaaagcatatgatgcagaaagatcgcggccgcgacgtcgattcacaaaaataggcacacgaaaaacaagtaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcg

SEQ ID NO: 13 - DNA, 7076 bp, Artificial sequence
  Integration vector pMOL2598
gatctaaagataatatctttgaattgtaacscccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatggccacattgaaaggggaggagaatcatgaaacaacaaaaacggctttacgcccgattgctgacgctgttatttgcgctcatcttcttgctgcctcattctgcagccgcggcacatcataatgggacaaatgggacgatgatgcaatactttgaatggcacttgcctaatgatgggaatcactggaatagattaagagatgatgctagtaatctaagaaatagaggtataaccgctatttggattccgcctgcctggaaagggacttcgcaaaatgatgtggggtatggagcctatgatctttatgatttaggggaatttaatcaaaaggggacggttcgtactaagtatgggacacgtagtcaattggagtctgccatccatgctttaaagaataatggcgttcaagtttatggggatgtagtgatgaaccataaaggaggagctgatgctacagaaaacgttcttgctgtcgaggtgaatccaaataaccggaatcaagaaatatctggggactacacaattgaggcttggactaagtttgattttccagggaggggtaatacatactcagactttaaatggcgttggtatcatttcgatggtgtagattgggatcaatcacgacaattccaaaatcgtatctacaaattccgaggtaaagcttgggattgggaagtagattcggaaaatggaaattatgattatttaatgtatgcagatgtagatatggatcatccggaggtagtaaatgagcttagaagatggggagaatggtatacaaatacattaaatcttgatggatttaggatcgatgcggtgaagcatattaaatatagctttacacgtgattggttgacccatgtaagaaacgcaacgggaaaagaaatgtttgctgttgctgaattttggaaaaatgatttaggtgccttggagaactatttaaataaaacaaactggaatcattctgtctttgatgtcccccttcattataatctttataacgcgtcaaatagtggaggcaactatgacatggcaaaacttcttaatggaacggttgttcaaaagcatccaatgcatgccgtaacttttgtggataatcacgattctcaacctggggaatcattagaatcatttgtacaagaatggtttaagccacttgcttatgcgcttattttaacaagagaacaaggctatccctctgtcttctatggtgactactatggaattccaacacatagtgtcccagcaatgaaagccaagattgatccaatcttagaggcgcgtcaaaattttgcatatggaacacaacatgattattttgaccatcataatataatcggatggacacgtgaaggaaataccacgcatcccaattcaggacttgcgactatcatgtcggatgggccagggggagagaaatggatgtacgtagggcaaaataaagcaggtcaagtttggcatgacataactggaaataaaccaggaacagttacgatcaatgcagatggatgggctaatttttcagtaaatggaggatctgtttccatttgggtgaaacgataatgctagcggccgcgtcgactagaagagcagagaggacggatttcctgaaggaaatccgtttttttattttgcccgtcttataaatttcgttgagatctacgcgtccatgggctagcgcggccgcgtcgacaggcctctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattattgcgacggggttgagaaaagccgattttgaagggctcatcaaggtttttgaaaagcgtgtcgttgtattcgggacgtcgattcacaaaaataggcacacgaaaaacaagtaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcgaattca

SEQ ID NO: 14 - DNA, 6972 bp, Artificial sequence
  Integration vector pMOL2606
gatctcaacgaaatttataagacgggcaaaataaaaaaacggatttccttcaggaaatccgtcctctctgctcttctagtcgacgcggccgctagcattatcgtttcacccaaatggaaacagatcctccatttactgaaaaattagcccatccatctgcattgatcgtaactgttcctggtttatttccagttatgtcatgccaaacttgacctgctttattttgccctacgtacatccatttctctccccctggcccatccgacatgatagtcgcaagtcctgaattgggatgcgtggtatttccttcacgtgtccatccgattatattatgatggtcaaaataatcatgttgtgttccatatgcaaaattttgacgcgcctctaagattggatcaatcttggctttcattgctgggacactatgtgttggaattccatagtagtcaccatagaagacagagggatagccttgttctcttgttaaaataagcgcataagcaagtggcttaaaccattcttgtacaaatgattctaatgattccccaggttgagaatcgtgattatccacaaaagttacggcatgcattggatgcttttgaacaaccgttccattaagaagttttgccatgtcatagttgcctccactatttgacgcgttataaagattataatgaagggggacatcaaagacagaatgattccagtttgttttatttaaatagttctccaaggcacctaaatcatttttccaaaattcagcaacagcaaacatttcttttcccgttgcgtttcttacatgggtcaaccaatcacgtgtaaagctatatttaatatgcttcaccgcatcgatcctaaatccatcaagatttaatgtatttgtataccattctccccatcttctaagctcatttactacctccggatgatccatatctacatctgcatacattaaataatcataatttccattttccgaatctacttcccaatcccaagctttacctcggaatttgtagatacgattttggaattgtcgtgattgatcccaatctacaccatcgaaatgataccaacgccatttaaagtctgagtatgtattacccctccctggaaaatcaaacttagtccaagcctcaattgtgtagtccccagatatttcttgattccggttatttggattcacctcgacagcaagaacgttttctgtagcatcagctcctcctttatggttcatcactacatccccataaacttgaacgccattattctttaaagcatggatggcagactccaattgactacgtgtcccatacttagtacgaaccgtccccttttgattaaattcccctaaatcataaagatcataggctccataccccacatcattttgcgaagtccctttccaggcaggcggaatccaaatagcggttatacctctatttcttagattactagcatcatctcttaatctattccagtgattcccatcattaggcaagtgccattcaaagtattgcatcatcgtcccatttgtcccattatgatgtgccgcggctgcagaatgaggcagcaagaagatgagcgcaaataacagcgtcagcaatcgggcgtaaagccgtttttgttgtttcatgattctcctcccctttcaatgtggccataatacataattttcaaactgataaaatgatttttcataaatccattagacggtgcaaatatatgtttttaatgttcttcgtttttaggcatccctcctttcaagataaataatttatacactattctattggaatcttaatcattccaatagaaaaatatgtaatgattataaataagtcgcttcttatcataaatatatttacatattcatttaatactacatcatgttaggtatagtaaggctatcaagggtgtcttaatttctacttgtaacaatgtattggcatattatatattgaattgagaaaattaaatacagcgataattcacatgaacaagttcattggtagttatattttcaaattttcaaggttgtgcttgtatgtcattctatagttagataagcatttgaggtagagtccgtccgaatatatttgtaatctgaagaaggttcaaacatatttctatataacgtattctttttttgtagttcttacttttgaggggsgttacaattcaaagatattatctttagatctggatcctctagagtcgacctgcaggcatgcaagcttgcatgcctgcaggtcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcgaattcaaaagccgcttccgccctggctttcgctttatccaaaggatgtgtcagccggttccacgcccggaacatcgtcccgtcgccgaaagggtctttcccgtccgcatcaaaggtatgccaatacgcaacagcgaagcgaaggtgttccttcatcgttttgccgccgacaaattcatcagggttgtaatatttaaatgcgtaaggattttccgaatcggccccttcatactcaatcattccgatatttctaaaaaacattccgatctcccccttcacttttccttgcaaacgttaaaaaacaatgtttgtttaaccattaaactaacttccttgtatgtattttacaggatcaattaatcgctttcaatggaaatagccgcggatcgatgctagca

SEQ ID NO: 15 - DNA, 1644 bp, Artificial sequence
  The bmy1 beta amylase-encoding gene
atgaaacaacaaaaacggctttacgcccgattgctgacgctgttatttgcgctcatcttcttgctgcctcattctgcagcagcggcgagcatagcaccaaatttcaaagtttttgtaatgggtccattagaaaaagtcacagattttaatgcattcaaagatcaattgataactttaaagaataatggtgtttatggtataacaacagatatttggtggggctatgttgaaaatgcaggtgaaaatcaatttgactggagttattataagacatatgctgataccgtacgcgctgcgggattgaagtgggttccaataatgtcaacgcatgcctgtggaggtaatgttggtgatacagtaaatatacctattccgtcatgggtatggacaaaagatacccaagataatatgcagtataaggatgaagccggaaattgggataatgaagcagtaagtccatggtattctggcttaacccaactctataatgaattttattcatcttttgcatcaaattttagcagctataaagatataattactaaaatatatatatctggaggcccttctggagaattaagatatccttcatataatccttcgcatggatggacatatcctggacgtggctcgctgcagtgctatagtaaagcggctataacaagttttcaaaatgctatgaagtctaaatatggaactatagcagcagttaatagtgcatggggtacaagcctaactgatttttctcaaattagtccacctacagatggtgataatttctttacaaatggttataaaactacttatggtaatgactttttgacatggtatcaaagtgttttgactaatgagttagccaatattgcttctgtagctcatagctgctttgatccagtatttaatgttccaataggagcaaaaatagctggagtgcattggctatataatagtccgacaatgccacatgctgcagaatattgtgccggttattataattatagcacgctactcgatcaatttaaggcatctaatcttgctatgacatttacatgtcttgaaatggatgattctaatgcatatgtaagtccatattattctgcacctatgacgttagtccattatgtagctaatcttgctaataataaaggtatagtccacaatggagaaaatgctttggctatatccaacaacaatcaagcttatgtgaattgtgcaaatgaattaacaggatataatttttctggatttacacttttaagactttcgaatattgtaaatagtgatggatctgtgacatcagagatggctccttttgtaattaatatagttacactaacgcctaacggtacgataccagttacatttacaataaacaatgcgacaacttattatggacaaaatgtatatattgttggtagtacatctgatcttggaaattggaatacaacctatgcccgtggtcctgcatcatgccctaattatcctacttggacaataacgcttaatctattacctggtgagcagatacagtttaaagctgtaaaaattgatagttcaggaaatgtaacttgggaaggtggctcgaatcatacttatactgtgccgacatctgggactggtagtgtcaccattacatggcaaaat

SEQ ID NO: 16 - DNA, 8170 bp, Artificial sequence
  Integration vector pAN369
aattcagatctaaagataatatctttgaattgtaacscccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatggccacattgaaaggggaggagaatcatgaaacaacaaaaacggctttacgcccgattgctgacgctgttatttgcgctcatcttcttgctgcctcattctgcagcagcggcgagcatagcaccaaatttcaaagtttttgtaatgggtccattagaaaaagtcacagattttaatgcattcaaagatcaattgataactttaaagaataatggtgtttatggtataacaacagatatttggtggggctatgttgaaaatgcaggtgaaaatcaatttgactggagttattataagacatatgctgataccgtacgcgctgcgggattgaagtgggttccaataatgtcaacgcatgcctgtggaggtaatgttggtgatacagtaaatatacctattccgtcatgggtatggacaaaagatacccaagataatatgcagtataaggatgaagccggaaattgggataatgaagcagtaagtccatggtattctggcttaacccaactctataatgaattttattcatcttttgcatcaaattttagcagctataaagatataattactaaaatatatatatctggaggcccttctggagaattaagatatccttcatataatccttcgcatggatggacatatcctggacgtggctcgctgcagtgctatagtaaagcggctataacaagttttcaaaatgctatgaagtctaaatatggaactatagcagcagttaatagtgcatggggtacaagcctaactgatttttctcaaattagtccacctacagatggtgataatttctttacaaatggttataaaactacttatggtaatgactttttgacatggtatcaaagtgttttgactaatgagttagccaatattgcttctgtagctcatagctgctttgatccagtatttaatgttccaataggagcaaaaatagctggagtgcattggctatataatagtccgacaatgccacatgctgcagaatattgtgccggttattataattatagcacgctactcgatcaatttaaggcatctaatcttgctatgacatttacatgtcttgaaatggatgattctaatgcatatgtaagtccatattattctgcacctatgacgttagtccattatgtagctaatcttgctaataataaaggtatagtccacaatggagaaaatgctttggctatatccaacaacaatcaagcttatgtgaattgtgcaaatgaattaacaggatataatttttctggatttacacttttaagactttcgaatattgtaaatagtgatggatctgtgacatcagagatggctccttttgtaattaatatagttacactaacgcctaacggtacgataccagttacatttacaataaacaatgcgacaacttattatggacaaaatgtatatattgttggtagtacatctgatcttggaaattggaatacaacctatgcccgtggtcctgcatcatgccctaattatcctacttggacaataacgcttaatctattacctggtgagcagatacagtttaaagctgtaaaaattgatagttcaggaaatgtaacttgggaaggtggctcgaatcatacttatactgtgccgacatctgggactggtagtgtcaccattacatggcaaaattaagcgcgcagctagctatgattaggagtgtttgcatttatgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagcggacgcactgaacatgcttgttcagcaaaaagtactcgatgataaatacgatgtctccgacaaagaaatcgacaaaaagctgaacgagtacaaaaaatcaatgggtgaccagctcaaccagctcattgaccaaaaaggcgaagacttcgtcaaagaacagatcaaatacgaacttctgatgcaaaaagccgcaaaggataacataaaagtaaccgatgatgacgtaaaagaatattatgacggcctgaaaggcaaaatccacttaagccacattcttgtgaaagaaaagaaaacggctgaagaagttgagaaaaagctgaaaaaaggcgaaaaattcgaagaccttgcaaaagagtattcaactgacggtacagccgaaaaaggcggcgacctcggctgggtcggcaaagacgataacatggacaaggatttcgtcaaagcggcatttgctttgaaaaccggcgaaatcagcggacctgtgaaatcccaattcggctatcacatcattaaaaaagacgaagaacgcggcaaatatgaagacatgaaaaaagagcttaaaaaagaagtccaagaacaaaagcaaaatgatcaaactgaactgcaatccgtcattgacaaacttgtcaaagatgctgatttaaaagtaaaagacaaagagttgaaaaaacaagtcgaccagcgtcaagctcagacaagcagcagcagctgacgccaaaaaagctgtcctcccctcgttggggtcggacagctttttttatgcgatggaatggctgtcagccgatttttcatgcggccgcgtcgactagaagagcagagaggacggatttcctgaaggaaatccgtttttttattttgcccgtcttataaatttcgttgagataactagtataagatctacgcgtccatgggctagcgcggccgcgtcgacaggcctctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattattgcgacggggttgagaaaagccgattttgaagggctcatcaaggtttttgaaaagcgtgtcgttgtattcgggacgtcgattcacaaaaataggcacacgaaaaacaagtaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcg

SEQ ID NO: 17 - DNA, 7938 bp, Artificial sequence
  Integration vector pAN405
aattcaaaagccgcttccgccctggctttcgctttatccaaaggatgtgtcagccggttccacgcccggaacatcgtcccgtcgccgaaagggtctttcccgtccgcatcaaaggtatgccaatacgcaacagcgaagcgaaggtgttccttcatcgttttgccgccgacaaattcatcagggttgtaatatttaaatgcgtaaggattttccgaatcggccccttcatactcaatcattccgatatttctaaaaaacattccgatctcccccttcacttttccttgcaaacgttaaaaaacaatgtttgtttaaccattaaactaacttccttgtatgtattttacaggatcaattaatcgctttcaatggaaatagccggatcgccgatgatcaagatctcaacgaaatttataagacgggcaaaataaaaaaacggatttccttcaggaaatccgtcctctctgctcttctagtcgacgcggccgctagctcagctgctgctgcttgtctgagcttgacgctggtcgacttgttttttcaactctttgtcttttacttttaaatcagcatctttgacaagtttgtcaatgacggattgcagttcagtttgatcattttgcttttgttcttggacttcttttttaagctcttttttcatgtcttcatatttgccgcgttcttcgtcttttttaatgatgtgatagccgaattgggatttcacaggtccgctgatttcgccggttttcaaagcaaatgccgctttgacgaaatccttgtccatgttatcgtctttgccgacccagccgaggtcgccgcctttttcggctgtaccgtcagttgaatactcttttgcaaggtcttcgaatttttcgccttttttcagctttttctcaacttcttcagccgttttcttttctttcacaagaatgtggcttaagtggattttgcctttcaggccgtcataatattcttttacgtcatcatcggttacttttatgttatcctttgcggctttttgcatcagaagttcgtatttgatctgttctttgacgaagtcttcgcctttttggtcaatgagctggttgagctggtcacccattgattttttgtactcgttcagctttttgtcgatttctttgtcggagacatcgtatttatcatcgagtactttttgctgaacaagcatgttcagtgcgtccgctccggcattgtcttttaatgtttgataaaggtcttcttttgtaatatttccagcttttgtttccgcaacaacctcagaatctcccccgctgcatgcgctgagagccagcacgcttgtcgctgtaatcgccgcaattgcaatcttcttcataaatgcaaacactcctaatcataacgcgttaattttgccatgtaatggtgacactaccagtcccagatgtcggcacagtataagtatgattcgagccaccttcccaagttacatttcctgaactatcaatttttacagctttaaactgtatctgctcaccaggtaatagattaagcgttattgtccaagtaggataattagggcatgatgcaggaccacgggcataggttgtattccaatttccaagatcagatgtactaccaacaatatatacattttgtccataataagttgtcgcattgtttattgtaaatgtaactggtatcgtaccgttaggcgttagtgtaactatattaattacaaaaggagccatctctgatgtcacagatccatcactatttacaatattcgaaagtcttaaaagtgtaaatccagaaaaattatatcctgttaattcatttgcacaattcacataagcttgattgttgttggatatagccaaagcattttctccattgtggactatacctttattattagcaagattagctacataatggactaacgtcataggtgcagaataatatggacttacatatgcattagaatcatccatttcaagacatgtaaatgtcatagcaagattagatgccttaaattgatcgagtagcgtgctataattataataaccggcacaatattctgcagcatgtggcattgtcggactattatatagccaatgcactccagctatttttgctcctattggaacattaaatactggatcaaagcagctatgagctacagaagcaatattggctaactcattagtcaaaacactttgataccatgtcaaaaagtcattaccataagtagttttataaccatttgtaaagaaattatcaccatctgtaggtggactaatttgagaaaaatcagttaggcttgtaccccatgcactattaactgctgctatagttccatatttagacttcatagcattttgaaaacttgttatagccgctttactatagcactgcagcgagccacgtccaggatatgtccatccatgcgaaggattatatgaaggatatcttaattctccagaagggcctccagatatatatattttagtaattatatctttatagctgctaaaatttgatgcaaaagatgaataaaattcattatagagttgggttaagccagaataccatggacttactgcttcattatcccaatttccggcttcatccttatactgcatattatcttgggtatcttttgtccatacccatgacggaataggtatatttactgtatcaccaacattacctccacaggcatgcgttgacattattggaacccacttcaatcccgcagcgcgtacggtatcagcatatgtcttataataactccagtcaaattgattttcacctgcattttcaacatagccccaccaaatatctgttgttataccataaacaccattattctttaaagttatcaattgatctttgaatgcattaaaatctgtgactttttctaatggacccattacaaaaactttgaaatttggtgctatgctcgccgctgctgcagaatgaggcagcaagaagatgagcgcaaataacagcgtcagcaatcgggcgtaaagccgtttttgttgtttcatgattctcctcccctttcaatgtggccataatacataattttcaaactgataaaatgatttttcataaatccattagacggtgcaaatatatgtttttaatgttcttcgtttttaggcatccctcctttcaagataaataatttatacactattctattggaatcttaatcattccaatagaaaaatatgtaatgattataaataagtcgcttcttatcataaatatatttacatattcatttaatactacatcatgttaggtatagtaaggctatcaagggtgtcttaatttctacttgtaacaatgtattggcatattatatattgaattgagaaaattaaatacagcgataattcacatgaacaagttcattggtagttatattttcaaattttcaaggttgtgcttgtatgtcattctatagttagataagcatttgaggtagagtccgtccgaatatatttgtaatctgaagaaggttcaaacatatttctatataacgtattctttttttgtagttcttacttttgaggggsgttacaattcaaagatattatctttagatctaagcttgcatgcctgcaggtcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcg